(19) United States
(12) Patent Application Publication
(10) Pub. No.: US 2003/0215454 A1
(43) Pub. Date: Nov. 20, 2003

(54) Binding of red blood cells to exposed subendothelial surfaces to impede platelet deposition thereon and/or for use in targeted drug delivery thereto

(76) Inventors: A. Mark Colb, West Roxbury, MA (US); Herman K. Gold, Brookline, MA (US)

Correspondence Address: KRIEGSMAN & KRIEGSMAN, 665 Franklin Street, Framingham, MA, 01702, US

(21) Appl. No.: 10/376,518

(22) Filed: Mar. 1, 2003

Related U.S. Application Data
(60) Provisional application No. 60/361,126, filed on Mar. 1, 2002.

(51) Int. Cl. A61K 39/395

(52) U.S. Cl. 424/175.1

(57) ABSTRACT

Binding of red blood cells (RBCs) to exposed subendothelial surfaces. According to one aspect of the invention, RBCs bind to a subendothelial surface that has been exposed by angioplasty so as to block the deposition of platelets onto the exposed surface, thereby impeding thrombosis and the triggering of restenosis by deposited platelets. A bispecific antibody is used to mediate the binding of RBCs to the exposed subendothelial surface, the bispecific antibody having a first antigen binding site directed against an RBC surface marker and a second antigen binding site directed against a subendothelial epitope. The bispecific antibody is preferably introduced into the bloodstream just prior to the performance of the angioplasty and is introduced in a quantity sufficient to bind a high percentage of RBCs. According to another aspect of the invention, RBCs are drawn from a patient, treated and then administered back to the patient for targeted drug delivery. The RBC treatment comprises coating the RBCs with two types of bispecific antibodies, the first type being adapted to bind the RBCs to an exposed subendothelial surface, the second type being adapted to removably bind the RBCs to a drug. The drug is then loaded onto the second type of bispecific antibody.

### CROSS-REFERENCE TO RELATED APPLICATIONS

The present application claims the benefit under 35 U.S.C. 119(e) of U.S. Provisional Patent Application Serial No. 60/361,126, filed Mar. 1, 2002, in the names of A. Mark Colb and Herman K. Gold, said provisional patent application being incorporated herein by reference.

### BACKGROUND OF THE INVENTION

The present application relates generally to the treatment of vascular disease and more particularly to the treatment of arterial atherosclerotic disease.

Atherosclerosis, which involves the deposition of a fatty plaque on the luminal surface of an artery, is one of the leading causes of death and disability in the world. This is because the deposition of plaque on the luminal surface of an artery causes progressive narrowing of the cross-sectional area of the artery. Such a narrowing reduces or blocks blood flow distal to the site of the lesion, causing ischemic damage to the tissue supplied by the artery.

The heart is supplied with blood via the coronary arteries. Consequently, narrowing of a coronary arterial lumen compromises the perfusion of heart muscle. This results in angina with exertion or even at rest. A complete occlusion of a vessel results in myocardial infarction, often causing death or subsequent heart failure. As is well known, the problem of coronary artherosclerosis is pervasive. There are over 1.5 million myocardial infarctions in the United States each year, resulting in the deaths of hundreds of thousands.

The preferred treatment for coronary atherosclerosis is percutaneous transluminal coronary balloon angioplasty (“PTCA”), with approximately one million such procedures performed each year in the United States alone. In PTCA, a balloon catheter is percutaneously inserted into a peripheral artery, threaded through the arterial system and then into the narrowed coronary artery to the site of the obstruction. The balloon is then inflated so as to expand radially outward, thereby crushing the plaque within the narrowed artery against the arterial wall and restoring the cross-sectional flow of blood through the treated coronary artery. Unfortunately, approximately 30-40% of those patients who undergo PTCA alone suffer from restenosis or a re-narrowing of the treated artery within six months of the procedure.

This restenosis is a response to local injury of the vessel wall caused by inflation of the balloon. Mechanisms of restenosis include (i) constrictive remodeling, likely due to retractile scar formation within the arterial wall, and (ii) the proliferation of smooth muscle cells with accompanying synthesis of extra-cellular matrix. This proliferation occurs in the intima, the layer beneath the inner lining of endothelial cells. The resulting thickening of the intimal layer (neointima) re-narrows the artery. See ea., Van Belle et al., “Endothelial regrowth after arterial injury: from vascular repair to therapeutics,” Cardiovascular Research, 38(1): 54-68 (April 1998), which is incorporated herein by reference. The use of stents at sites of angioplasty has reduced the rate of restenosis to 20-25%. This remaining incidence is due principally to neointimal proliferation.

It is believed that this intimal thickening is elicited by platelet adherence to the exposed subendothelium at the site of injury and subsequent activation of the adherent platelets to release the contents of their alpha granules. These granules contain platelet-derived growth factor (PDGF), a chemotactic attractant and a very strong mitogen for smooth muscles cells. The diffusion of PDGF and like factors into the intimal layer stimulates the migration of smooth muscle cells into the neointima and drives their subsequent proliferation. See e.g., Chandrasekar et al., J. Am. Coll. Cardiol., 35(3):555-62 (2000); and Banters et al., Prog. Cardiovasc. Dis., 40(2):107-16 (1997), both of which are incorporated herein by reference.

Evidence supporting the role of platelet deposition in the development of restenosis includes the following:

Platelet adherence to the subendothelium at the injury site is an early event in a variety of models of angioplasty. For instance, within 30 minutes of balloon injury to the rabbit iliac artery or aorta, the denuded intima is covered with platelets which have spread and degranulated. See Stemerman, Am. J. Pathol., 63:7-26(1973); Wilentzet al., Circulation, 75(3):636-42 (1987); Groves et al., Lab. Invest., 40(2):194-200 (1979), all of which are incorporated herein by reference. In addition, pathology of stented human vessels shows dense platelet deposition on the struts of stents placed days-to-weeks before death. See Farb, Circulation, 99:44-52 (1999), which is incorporated herein by reference.

In the rabbit model of arterial injury, thrombocytopenia inhibits neointimal thickening. The degree of inhibition is related to the severity of the thrombocytopenia. See Chandrasekar et al., J. Am. Coll. Cardiol., 35(3):555-62 (2000).

Abnormally high platelet reactivity is associated with a 2-3 fold higher rate of restenosis. See Chandrasekar et al., J. Am. Coll. Cardiol., 35(3):555-62 (2000).

In a canine model of coronary injury, cyclic variations in blood flow occur. Typically, flow declines over some period and is then abruptly restored. These flow variations correspond to cycles of platelet accumulation and sudden dislodgment. It is reported that the severity of subsequent neointimal proliferation is closely related to the frequency and severity of these cyclic flow variations during the week after injury. Hence, the neointimal reaction is correlated with antecedent platelet deposition. Aggressive anti-platelet treatment eliminates the flow variations, also minimizing neointimal thickening. See Willerson et al., PNAS, 88:10624-8 (1991), which is incorporated herein by reference.

Oligonucleotide antisense to the PDGF receptor was delivered locally to injured rat carotid artery, inhibiting expression of the PDGF receptor. As a result, initimal thickening was dramatically reduced. A strong correlation was observed between the residual level of receptor expression and the extent of neointimal proliferation. See Sirois et al., Circulation, 95:669-76 (1997).

In keeping with this view that platelet deposition at an angioplasty injury site plays an important role in restenosis, a number of anti-platelet agents have been tried in an effort to reduce restenosis after angioplasty. Such agents have included aspirin, ticlopidine, IIb/IIIa inhibitors (e.g., integrilin) and others. At present, none has shown a significant benefit. See Lelkovits et al., Prog. Cardiovasc. Dis., 40:141-58 (1997), which is incorporated herein by reference.

These anti-platelet agents inhibit aggregation of platelets, but do not prevent platelet adherence to a site of injury. For instance, it has been observed that abciximab, a potent IIb/IIIa inhibitor in wide clinical use, does not prevent deposition of a monolayer of platelets at a site of experimental angioplasty in monkeys. See Palmerini et al., J. Am. Coll. Cardiol., 40:360-6 (2002), which is incorporated by reference. A monolayer of adherent platelets may be quite sufficient to give the initial stimulus that elicits intimal hyperplasia.

Another complication of angioplasty is subacute thrombosis, occurring within days following the procedure. The incidence of thrombosis, although low, is still significant, particularly in certain groups, including diabetics, patients with small vessel diameters, and patients undergoing multi-vessel procedures. In these groups, the rate of thrombosis reaches 3% or more, See Reynolds et al., J. Invas. Cardiol., 14:364-8 (2002), which is incorporated herein by reference.

Moreover, the low general rate of thrombosis is largely the result of anti-platelet therapy, as with IIb/IIIa inhibitors, which creates a significant risk of bleeding. Such therapy poisons platelet function systemically, hence complicating or precluding its use in patients at high risk of bleeding, especially intra-cerebral bleeding.

In view of the above, it can be readily appreciated that there is a definite need for a technique to prevent platelet deposition at an angioplasty site. This would serve the dual purpose of preventing thrombosis and restenosis. Preferably, such a technique would not impair the normal function of platelets elsewhere in the body.

### SUMMARY OF THE INVENTION

It is an object of the present invention to provide a novel technique for impeding the deposition of platelets onto an exposed subendothelial surface, which exposed surface may be present, for example, following the performance of a balloon angioplasty on an artery.

In accordance with the teachings of the present invention, said technique involves binding red blood cells to the exposed subendothelial surface, thereby forming a coating or shield thereover to prevent the deposition of platelets onto the subendothelial surface. This may be done, according to a first embodiment, by introducing into the bloodstream of the patient a quantity of a multispecific antibody, said multispecific antibody comprising a first antigen binding site and a second antigen binding site, said first antigen binding site being directed against a surface marker of red blood cells (RBCs), said second antigen binding site being directed against a subendothelial epitope. The multispecific antibody is preferably introduced into the bloodstream just prior to the performance of the angioplasty and is introduced in a quantity sufficient to bind a high percentage of RBCs. In this manner, once the angioplasty has been performed and the target epitopes on the subendothelium have been exposed, the multispecific antibodies that have already bound the RBCs then bind the RBCs to the subendothelium. Thus covered by the bound RBCs, the previously exposed subendothelium is no longer accessible for platelet deposition. In this manner, by impairing platelet deposition onto the subendothelium, intimal thickening (and, ultimately, restenosis) triggered by platelet deposition may be inhibited. Since platelet deposition is the initial step in clotting, thrombosis of the angioplasty site is also prevented.

The present invention is also directed to a technique for targeting drug delivery to exposed subendothelial surfaces, which surfaces may be present, for example, following the performance of a balloon angioplasty on an artery, or as a result of other vascular disease (e.g., vasculitis, transplant arteriosclerosis). Said targeted drug delivery may be accomplished, according to a first embodiment, by introducing into the bloodstream of the patient a quantity of treated red blood cells (RBCs), the treated RBCs being adapted to bind to exposed subendothelial surfaces and having a therapeutic agent removably coupled thereto. Preferably, a first multispecific antibody is used to bind the treated RBCs to exposed subendothelial surfaces, and a second multispecific antibody is used to bind the therapeutic agent to the treated RBCs. More specifically, the first multispecific antibody preferably comprises a first antigen binding site and a second antigen binding site, the first antigen binding site being directed against a surface marker of RBCs, the second antigen binding site being directed against a subendothelial epitope. The second multispecific antibody preferably comprises a first antigen binding site and a second antigen binding site, the first antigen binding site being directed against a surface marker of RBCs, the second antigen binding site being directed against the therapeutic agent.

The treated RBCs may be introduced into the bloodstream at the time of an angioplasty, for example. The shape of an RBC is a biconcave disk with pronounced concavities. Therefore, when the treated RBCs bind to the exposed subendothelium, a small volume is enclosed between the bound RBC and the underlying subendothelium. A portion of the bound therapeutic agent quickly dissociates from each treated RBC into the aforementioned volume until an equilibrium concentration is reached. As the quantity of dissociated therapeutic agent in said volume is depleted by diffusion into the subendothelium, additional therapeutic agent dissociates from the treated RBC, maintaining the equilibrium. In this manner, a therapeutic concentration of the therapeutic agent can be applied to the desired site for an extended period of time. The agent is applied to the entire surface of injury under a monolayer of adherent RBCs. This is accomplished in the absence of any significant plasma level of the agent. (The plasma is, in effect, a separate compartment.) The treatment continues so long as there is an excess of bound agent on the overlying RBC surfaces and the RBCs remain adherent.

Additional objects, features, aspects and advantages of the present invention will be set forth, in part, in the description which follows and, in part, will be obvious from the description or may be learned by practice of the invention. In the description, reference is made to the accompanying drawings which form a part thereof and in which is shown by way of illustration specific embodiments for practicing the invention. These embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate preferred embodiments of the invention and, together with the description, serve to explain the principles of the invention. In the drawings wherein like reference characters represent like parts:

FIGS. 1(a) through 1(c) are schematic views illustrating the attachment of red blood cells to an exposed subendothelial surface so as to shield the subendothelial surface against platelet deposition in accordance with the teachings of the present invention (FIGS. 1(a) through 1(c) not being drawn to scale); and

FIGS. 2(a) through 2(c) are schematic views illustrating the targeted delivery of a therapeutic agent to an exposed subendothelial surface in accordance with the teachings of the present invention (FIGS. 2(a) through 2(c) not being drawn to scale).

### DETAILED DESCRIPTION OF PREFERRED EMBODIMENTS

The present invention is directed to a technique for binding red blood cells (RBCs) to exposed subendothelial surfaces. A first application of the technique is in the formation of an RBC monolayer over newly exposed subendothelial surfaces (such as may be presented after the performance of an angioplasty) in order to physically shield such surfaces against platelet deposition. By shielding such exposed endothelial surfaces from platelet deposition for a period of time (on the order of 24 hours), one may prevent platelet deposition altogether since the exposed luminal surface is known to lose its adhesiveness for platelets well within this time frame. (See, for example, Groves et al., Arteriosclerosis, 6:189-95 (1986), which is incorporated herein by reference.) One may then prevent both events that are initiated by platelet deposition on the subendothelium, thrombosis and intimal hyperplasia. A second application of the technique is in the targeted delivery of therapeutic agents to exposed subendothelial surfaces, using RBCs as drug delivery vehicles.

The formation of a platelet shield of the type described above is accomplished, according to one embodiment, by introducing into the bloodstream of a patient, e.g., by injection, a quantity of a multispecific antibody, said multispecific antibody comprising a first antigen binding site and a second antigen binding site, said first antigen binding site being directed against a surface marker of RBCs, said second antigen binding site being directed against a subendothelial epitope. The multispecific antibody is preferably introduced into the bloodstream just prior to performance of the angioplasty (but may also be introduced during or directly after the angioplasty) and is introduced in a quantity sufficient to bind a high percentage of circulating RBCs. In this manner, once the angioplasty has been performed and the target epitopes on the subendothelium have been exposed, the multispecific antibodies that have already become bound to the circulating RBCs also then rapidly bind to the subendothelium. Thus quickly covered by the bound RBCs, the previously exposed subendothelium is rendered substantially inaccessible to deposition by platelets.

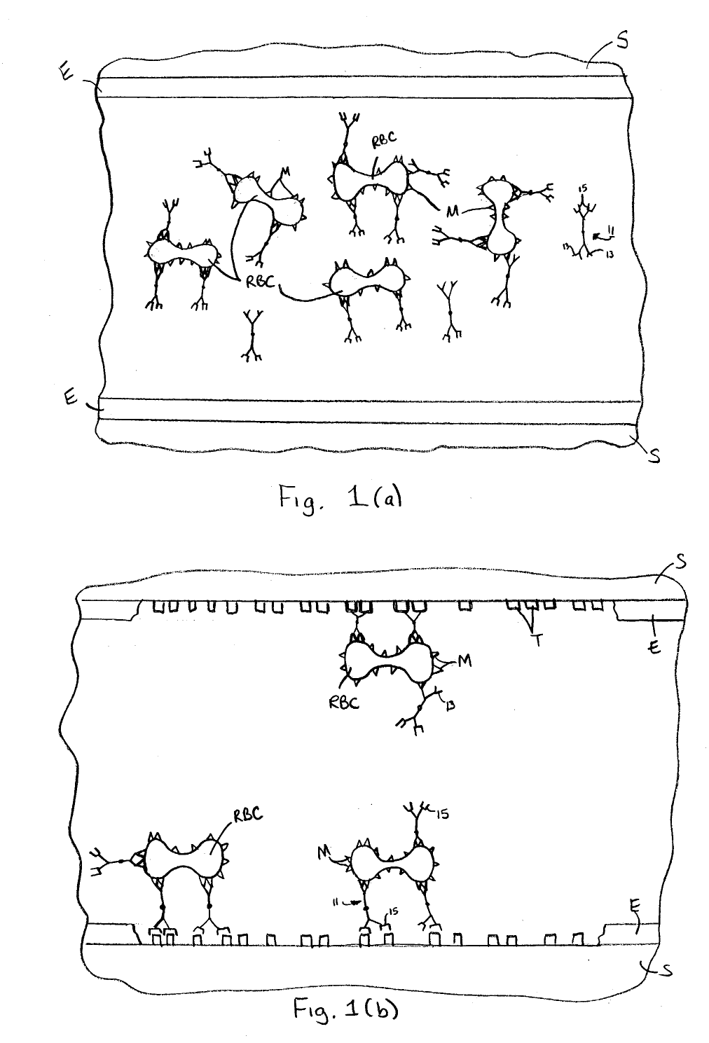

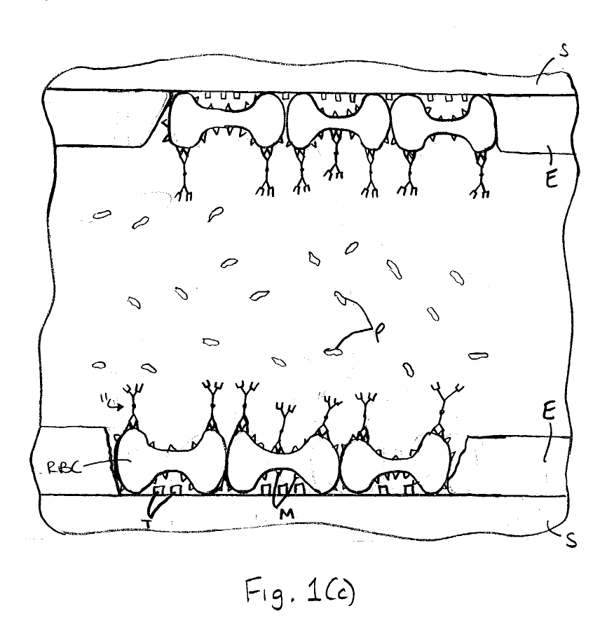

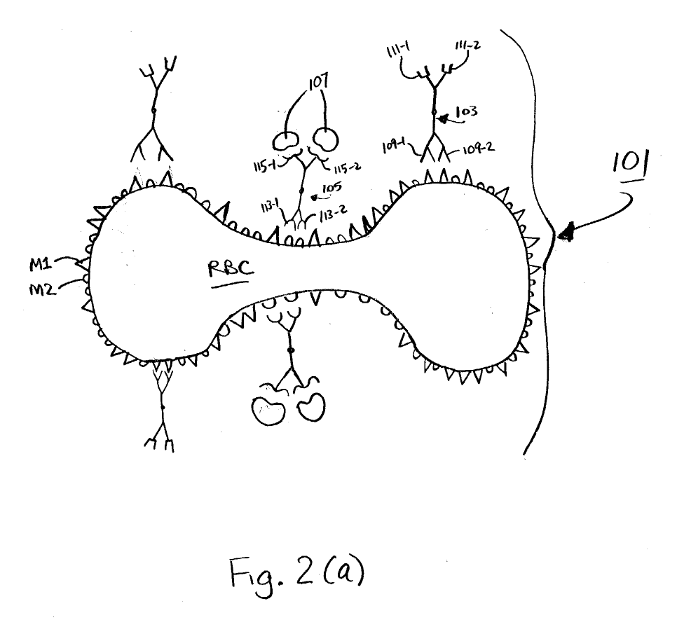

Referring now to FIGS. 1(a) through 1(c), there is shown a series of schematic views illustrating generally the platelet shielding technique described above. In FIG. 1(a), prior to an angioplasty being performed (the plaque not shown), a quantity of a bispecific antibody 11 is injected into a patient's bloodstream, each such bispecific antibody 11 comprising a pair of first antigen binding sites 13 and a pair of second antigen binding sites 15, said bispecific antibodies 11 binding via their first antigen binding sites 13 to surface markers M on red blood cells RBC. In FIG. 1(b), as a result of the angioplasty being performed, a portion of the endothelial layer E of the artery is stripped, exposing target epitopes T in the subendothelial matrix S to which red blood cells RBC begin to bind through antibodies 11. In FIG. 1(c), a tiling or monolayer of red blood cells RBC is formed over the entirety of the previously exposed area of the subendothelial matrix S, said tiling serving to prevent platelets P from being deposited directly onto the subendothelial matrix S. (For clarity, the antibodies 11 binding the red blood cells RBCs to the subendothelial matrix S are not shown in FIG. 1(c).)

The present inventors believe that the foregoing technique results in the nearly instantaneous formation of an RBC monolayer or shield over any exposed subendothelial surface, thereby preventing or minimizing the direct deposition of platelets onto the exposed subendothelium. It should be noted that the antibody-coated RBCs have a large competitive advantage over platelets in binding to the subendothelium as the ratio of RBCs to platelets in blood is approximately 20 to 1. Moreover, RBCs are larger than platelets by orders of magnitude; therefore, each RBC binding event covers far more surface area than would be the case for a platelet. In addition, the number of binding sites per RBC and their affinity for target epitopes can be optimized to enhance the competitive advantage to any desired degree.

As a result of the present method, RBCs coat the injured surface almost completely, lying flat over the exposed surface and leaving only small interstices between contiguous red cells. The flat position (FIG. 1(c)), with the circular rim of the RBC parallel to the luminal surface, maximizes points of contact between antibodies coating the RBC and their target epitopes on the subendothelial surface. This position also minimizes the exposure of an adherent RBC to stripping forces associated with blood flow. Hence, this is the configuration that will be most favored and assumed by the adherent RBCs. The maximum attainable coverage, based on a planar model, is π(2(3)^{0.5}), or slightly over 90 percent of exposed subendothelium. If bound RBCs are stripped from the subendothelium at some rate by shear forces, they are instantly replaced by other RBCs. The coating capacity should be fairly long lived and, in any event, need only be for as long as exposed subendothelial surfaces retain their adhesiveness to platelets. The latter period is under 12 hours in experimental models. Antibodies coating the RBCs will be gradually lost over a period of days, in keeping with known kinetics. The coating of the subendothelium will then also be lost, but the benefit will have already accrued.

A significant advantage over conventional anti-platelet therapy aimed at preventing thrombosis at the angioplasty site is that the present method does not poison platelet function. The method is unique in this respect, among anti-platelet therapies. It leaves intrinsic platelet function completely unimpaired so that clotting may occur normally at sites of bleeding.

The RBC blockade of platelet deposition operates within the angioplasty site, which is a discrete 2-dimensional surface that can be readily covered within its borders. At a site of bleeding, however, platelets flow into an open tissue space with 3-dimensional geometry and multiple surfaces available for platelet attachment. In this milieu, it is extremely unlikely that platelet adherence can be blocked. The subsequent events of platelet aggregation and clotting activation should then occur normally.

As a result, this method can be offered to angioplasty patients who are not candidates for existing types of anti-platelet therapy because of the associated risk of bleeding.

This method may also prevent thrombosis following angioplasty in those cases where the risk of thrombosis is still significant. Such cases include diabetics, angioplasty of arteries of small diameter, and multi-vessel angioplasty.

Many different antigens on the RBC surface can serve as the cell surface marker against which the first antigen binding site of the aforementioned multispecific antibody may be directed. One such antigen is the D antigen of the Rh blood group. The D antigen, which includes multiple epitopes, is an attractive choice for several reasons, namely, it is present in over 80% of individuals, its expression is limited to erythroid cells, and its copy number is substantial (greater than 10⁴/cell). Other attractive choices include glycophorins A and B, which are RBC membrane glycoproteins having very high copy numbers (10⁶/cell in the case of glycophorin A and 10⁵/cell in the case of glycophorin B). See also Poole, Blood Reviews, 14:31-43 (2000), which is incorporated herein by reference. It is worth noting that the rare individuals lacking glycophorin A on RBCs suffer no significant consequences. Hence, the coating of a fraction of glycophorin A molecules with antibody should be well tolerated. Another surface antigen of high copy number is B and 3.

Examples of suitable subendothelial components against which the second antigen binding site may be directed include collagen (especially types 1 and 3), elastin, laminin, and fibronectin. The interior of a plaque is rich in collagen and other proteinaceous components of connective tissue matrix. (Virmani et al., Arterioscler. Thromb. Vasc. Biol. 20:1262-75 (2000), which is incorporated herein by reference.) Hence, the exposed interior can be targeted along with subendothelium by the same antigen binding site. (The typical angioplasty exposes both subendothelium and plaque interior.) Antibody clones directed at other subendothelial epitopes can be isolated, preferably by phage display technology, using human arterial specimens in the screening.

Multispecific antibodies for use in the above-described technique may be prepared, for example, by any means known in the art including, but not limited to, those techniques disclosed in U.S. Pat. No. 6,458,933; U.S. Pat. No. 4,714,681; U.S. Pat. No. 4,444,878; and U.S. Pat. No. 4,331,647, as well as in Wickham et al., J. Virol., 70(10):6831-8 (1996), all of which are incorporated herein by reference. Such multispecific antibodies may comprise two or more intact antibodies that are covalently bound to one another or may comprise two or more antibody fragments, e.g., Fab′, F(ab′)₂, Fᵥ, that are covalently bound to one another. It is important to note that these fragments all lack the F_{c} portion of the intact antibody molecule. Hence, red blood cells coated with such fragments will escape rapid clearance by the RES (reticuloendothelial system). Such clearance is mediated by the F_{c} receptors of macrophages and macrophage-like cells of the RES.

For purposes of the present specification and claims, the term “antibody,” unless specifically limited otherwise, shall be construed broadly enough to encompass any molecule containing an antigen-binding site derived from an antibody, either directly or through subcloning a DNA fragment encoding the site. Each antibody fragment of the subject multispecific antibody may be monovalent (i.e., containing one antigen binding site) or multivalent (i.e., containing a plurality of antigen binding sites). Each such antibody fragment may have a similar or dissimilar valence to another such antibody fragment. Components of the multispecific antibody may be prepared from monoclonal antibodies or from polyclonal antibodies. Fragments may be derived from specific digestion (e.g., with papain or pepsin), reductive cleavage of disulfide bonds, or by other treatment of antibody molecules, methods for which are well-established. Fragments may also be derived through subcloning of DNA fragments encoding the antigen binding site into appropriate vectors that permit expression in prokaryotic or eukaryotic cells. Methods for deriving such fragments are also well-known in the art.

Representative techniques for preparing bispecific antibodies are as follows: Begin with pure preparations of two different monoclonal antibodies (Mab). One Mab is reacted with SATA (N-succinimidyl S-acetylthioacetate). The product is then deprotected by treatment with hydroxylamine to yield an SH-Mab, the antibody now containing free sulfhydryl groups. The second Mab is reacted with sSMCC (sulfosuccinimidyl 4-(N-maleimidomethyl)cyclohexane-1-carboxylate). The respective reactions products SH-Mab and sSMCC-Mab are purified by gel filtration under argon, and then reacted together. The product of this coupling reaction is the desired conjugated bispecific antibody, which is then purified by gel filtration. Details of the procedure are given in Lindorfer et al., J. Immunol., 167:2240-9 (2001), which is incorporated herein by reference.

Digestion of an IgG antibody molecule with pepsin releases an F(ab′)₂ fragment containing two antigen-binding sites linked by a disulfide bond between the two heavy chains. This bond can be cleaved by reduction releasing two identical Fab′ fragments containing the binding sites. These Fab′ fragments can be mixed with the Fab′ fragments derived from a second Mab, and disulfide linkages then reformed by oxidation. Among the products there will be bispecific F(ab′)₂ fragments with one Fab from each of the original Mabs. This bispecific product can then be chromatographically purified.

Alternatively, Fab′ fragments of one specificity (derived from one Mab) can be activated with an excess of bis-maleimide linker (1,1′-(methylenedi-4,1-phenylene)bis-maleimide). Fab′ fragments of a second Mab (released by reduction of F(ab′)₂ fragments), can then be reacted with the activated Fab′ fragments of the first Mab to give a high yield of bispecific molecules.

It is also possible to produce bispecific antibodies through cell fusion of two hybridoma cells secreting the respective Mabs. These so-called hybrid-hybridomas can be selected in culture by standard means, and then screened for the production of both antibodies. Bispecific antibody molecules will be among the secreted products, along with bivalent antibody of the two ‘parental’ types. The bispecific molecules can then be purified by hydrophobic interaction chromatography. (See Weiner et al., J. Immunol., 147:4035-44 (1991), which is incorporated herein by reference.)

Recombinant DNA technology can also be utilized in the preparation of bispecific antibody fragments. The Fᵥ fragment contains the antigen binding site of the antibody. It consists of the V_{L} and V_{H} subfragments in noncovalent association. If a peptide linker is interposed between them covalently, a fusion protein results, known as an SCFᵥ (single chain variable fragment). The SCFᵥ can bind the target epitope. DNA encoding an SCFᵥ, or more than one, can be subcloned into a vector that contains all necessary regulatory elements to permit expression in a prokaryotic or a eukaryotic cell. This host cell then produces the desired bispecific molecule.

Certain of these and other established methods are readily adaptable to the preparation of higher order molecules containing additional binding sites for the same two or for additional target epitopes.

The necessary Mabs directed at the various targets identified above, and contributing binding sites to multispecific antibodies, are available. The Mabs of nonhuman origin can be ‘humanized’ by well established methods. In addition, other Mabs directed at these targets can be readily isolated using methods that are routine in the practice of the art.

Additional monoclonals directed at subendothelial targets can be isolated so as to ensure binding to the intact native structure and not simply to purified components. Lengths of denuded artery with exposed subendothelium can be used in the screening by phage display technology. In this way, the isolated clones will recognize target structures as they will appear in vivo during treatment, configured and assembled, and not simply in the form of pure components.

To enhance the targeting of the subendothelium, one may use a cocktail of bispecific antibodies differing in their respective second antigen binding sites so as to be directed against different subendothelial connective tissue components. Alternatively, one may use multispecific antibodies having, in addition to one or more same or different RBC binding sites, a plurality of different subendothelial binding sites. Available techniques, cited above, permit the conjugation of multiple fragments, yielding antibody molecules with multiple and diverse binding sites.

In addition to shielding an exposed subendothelial surface against platelet deposition, one may also shield a stent against platelet deposition by coating the stent, prior to its implantation within an artery, with antibodies (or fragments thereof that are directed against RBCs. In this manner, after the stent is deployed, it quickly becomes coated with RBCs. Alternatively, instead of coating the stent with an anti-RBC antibody, one could biotinylate the stent and administer to the patient an anti-RBC antibody conjugated with avidin. In this manner, RBCs become coated onto the stent through an avidin-biotin complex.

As noted above, the present invention is also directed to the targeted delivery of therapeutic agents to exposed subendothelial surfaces using RBCs as drug delivery vehicles.

This is accomplished, according to a first embodiment of said technique, by introducing into the bloodstream of the patient a quantity of treated red blood cells (RBCs), the treated RBCs being adapted to bind to exposed subendothelial surfaces and having a therapeutic agent removably bound thereto. Preferably, a first multispecific antibody is used to bind the treated RBCs to exposed subendothelial surfaces, and a second multispecific antibody is used to bind the therapeutic agent to the treated RBCs. More specifically, the first multispecific antibody preferably comprises a first antigen binding site and a second antigen binding site, the first antigen binding site being directed against a surface marker of RBCs, the second antigen binding site being directed against a subendothelial epitope. The second multispecific antibody preferably comprises a first antigen binding site and a second antigen binding site, the first antigen binding site being directed against a surface marker of RBCs, the second antigen binding site being directed against the therapeutic agent.

The treated RBCs are preferably obtained by drawing a blood sample from a patient, e.g., 10 ml, adding the first and second multispecific antibodies to the blood to permit the coating of the RBCs with the first and second multispecific antibodies, and then adding the therapeutic agent to the antibody-coated RBCs to permit the binding of the therapeutic agent to the second multispecific antibody.

The treated RBCs are then introduced into the bloodstream of the patient at the time of an angioplasty, for example, with the aim of preventing restenosis. Because the shape of an RBC is a biconcave disk, when the treated RBCs bind to the subendothelium, a small volume is enclosed between each adherent RBC and the underlying subendothelium. This volume is, in effect, a compartment separate from the surrounding plasma, essentially sealed off from it with respect to the diffusion of large molecules. The compartment is kept separate for as long as the RBC adheres.

A portion of the bound therapeutic agent quickly dissociates from each adherent RBC into the aforementioned volume until an equilibrium concentration is reached with the bound fraction. As the quantity of dissociated therapeutic agent in said volume is depleted by diffusion into the subendothelium, the intended site of action, additional therapeutic agent dissociates from the overlying RBC, maintaining equilibrium between the free and bound fractions. In this manner, a therapeutic concentration of the therapeutic agent can be delivered to the desired site for an extended period of time. The agent is thus applied to the arterial surface over the entire area of injury, under a monolayer of adherent RBCs. At no time is there a significant plasma level of the agent.

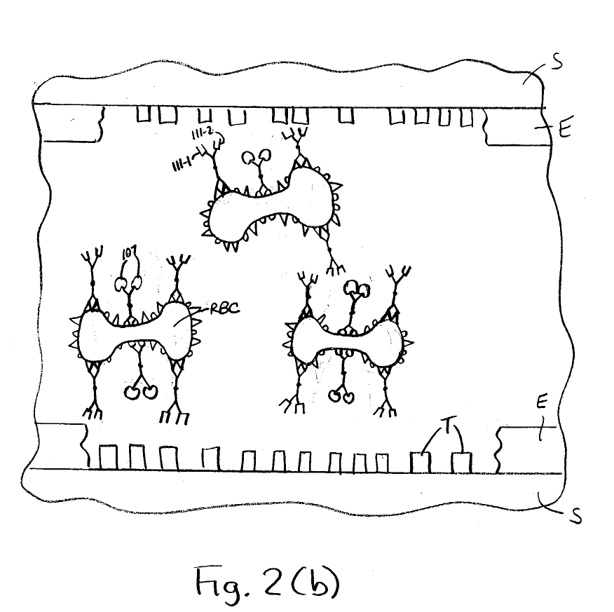

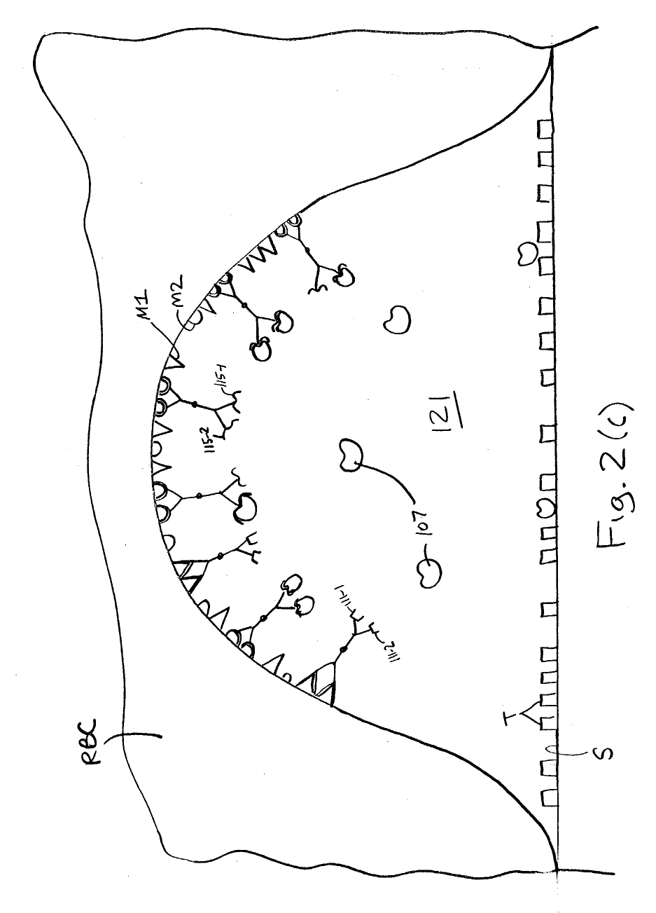

Referring now to FIGS. 2(a) through 2(c), there is shown a series of schematic views illustrating the targeted delivery of a therapeutic agent to an exposed subendothelial surface in accordance with the teachings of the present invention. FIG. 2(a) is an exploded view of a treated red blood cell 101 prior to its administration to a patient, the treated red blood cell 101 comprising a red blood cell RBC from the patient, first and second bispecific antibodies 103 and 105, respectively, and a therapeutic agent 107. As can be seen, cell surface markers M1 and M2 are dispersed over the surface of red blood cell RBC. First bispecific antibody 103 is bound to red blood cell RBC through a pair of first antigen binding sites 109-1 and 109-2 directed against markers M1, antibody 103 also having a pair of second antigen binding sites 111-1 and 111-2 directed against a subendothelial epitope. Second bispecific antibody 105 is bound to red blood cell RBC through a pair of first antigen binding sites 113-1 and 113-2 directed against markers M2, antibody 105 also having a pair of second antigen binding sites 115-1 and 115-2 directed against therapeutic agent 107. In FIG. 2(b), a quantity of treated red blood cells 101 are injected into a patient's bloodstream at about the time an angioplasty is performed. (For clarity, the plaque in the patient's vessel is not shown). As can be seen, as a result of the angioplasty, a portion of the endothelium E is stripped, exposing epitopes T in the subendothelium S. The exposure of epitopes T in subendothelium S allows for the binding of treated red blood cells 101 to subendothelium S. In FIG. 2(c), which is an enlarged fragmentary view of a treated red blood cell 101 bound to the subendothelium S, it can be seen that, because of the biconcave shape of red blood cell RBC, a substantially closed volume 121 is formed between red blood cell RBC and the subendothelium S. (For clarity, the antibodies 103 binding red blood cell RBC to the subendothelium S are not shown.) A quantity of therapeutic agent 107 dissociates from antibody 105 into volume 121 until an equilibrium concentration is reached. As the quantity of dissociated therapeutic agent 107 in volume 121 is depleted by diffusion into subendothelium S, additional therapeutic agent 107 dissociates from antibody 105. In this manner, a steady therapeutic concentration of agent 107 can be maintained in volume 121 for a substantial period of time, even without a significant concentration of agent 107 outside of volume 121, i.e., in the blood plasma.

It should be noted that, whereas antigen binding sites 109-1/109-2 and 113-1/113-2 are shown in FIGS. 2(a) through 2(c) as being directed to two different markers M1 and M2, respectively, they could be directed to the same marker.

The following is offered in further illustration of the invention: Suppose that one wishes to apply an agent to a stripped arterial wall at a concentration of 10⁻⁷ M. Assume that the volume trapped beneath an adherent RBC is roughly equal to the RBC volume itself, about 10⁻¹³ liters. A concentration of 10⁻⁷ M then requires 6000 free molecules in the trapped volume. Let the K_{D} of the antibody binding site for the ligand (agent) also be 10⁻⁷ M. Then, a free ligand concentration of 10⁻⁷ M is associated, at equilibrium, with 50% occupancy of binding sites.

If, at the initial equilibrium, 50,000 molecules of ligand remain bound to the overlying RBC surface, that excess is a sufficient store for extended repletion of the compartment. The bound 50,000 molecules, representing 50% occupancy, suggests a total of 100,000 binding sites on that face of the RBC, or 200,000 in all per RBC. This is readily achievable with glycophorin A as the attachment site on the RBC surface, with its 10⁶ molecules per cell. Alternatively, heteropolymeric molecules containing many ligand-binding sites per molecule can be bound to the RBCs at a smaller number of sites.

An aliquot of the patient's blood is taken and coated with bispecific antibody to give 200,000 ligand-binding sites per cell. The RBCs are then loaded with ligand ex ViVO at a concentration slightly above the K_{D}. The RBCs are infused at the time of angioplasty. It is desirable that the off-rate for the antibody-bound ligand be slow so that a minimal amount of ligand is lost by dissociation in the brief interval prior to RBC binding at the angioplasty site. The subendothelial sites are exposed by the angioplasty, the loaded RBCs bind and elute the ligand to an initial equilibrium concentration of 10⁻M as specified above. (If the loading of RBCs with ligand is done at a higher concentration, the initial equilibrium concentration in the trapped volume will be higher.) The foregoing is only a rough example. Clearly, the parameters can be chosen within a considerable range to optimize the result. Ligand concentrations of 10⁻⁶ M and higher should be readily achievable beneath adherent RBCs. This is comparable to the plasma concentration achieved for many drugs given systemically. For example, a 10 mg does of an agent with a molecular weight of 1000 is equal to 10⁻⁵ moles. Distributed in the plasma and extravascular fluid volume, totalling roughly 20 liters, this gives a concentration of 0.5×10⁻⁶ M. It is also noteworthy that hepatocyte growth factor, for instance, a potent endothelial growth factor, has a K_{D} of 0.35×10⁻⁹ M for its receptor. (Bussolino et al., J. Cell. Biol., 119:629-41 (1992), which is incorporated herein by reference.)

Examples of therapeutic agents usable in the above-described technique include growth factors for promoting endothelialization, cytotoxic or cytostatic agents for inhibiting cell proliferation in the neointima and immunosuppressive agents.

The above-described technique is not limited to use with subendothelial surfaces that are exposed by angioplasty, with the purpose of preventing restenosis. Of at least equal importance are the prospects for treatment of a variety of vascular diseases with the shared characteristic that endothelial cells are shed from the luminal surface of involved blood vessels at sites of active disease. These include various forms of vasculitis, both primary and secondary to a collagen vascular disease such as lupus or rheumatoid arthritis.

It has been reported that endothelial cells are typically detached in small vessel vasculitis. (Woywodt et al., Lancet, 361:206-10 (2003), which is incorporated herein by reference.) As a result, large numbers of circulating endothelial cells are detected in affected subjects. Similarly, it has been reported that the numbers of circulating endothelial cells are much elevated in patients with active SLE (systemic lupus erythematosis), due presumably to ongoing vasculitis. (Clancy et al., Arthritis Rheum., 44:1203-8 (2001), which is incorporated herein by reference.)

Transplant arteriosclerosis is a diffuse intimal hyperplasia in the vessels of an organ graft. It is a very important clinical problem, limiting graft survival. In experimental models it has been shown that the endothelial cells lining the vessels of the graft are lost and replaced by host cells. (Hillebrands et al., J. Clin. Invest., 107:1411-22 (2001), which is incorporated herein by reference.)

In all these conditions, the local loss of endothelial cells exposes subendothelium, to which RBCs may be targeted. The RBCs are then “smart vehicles,” delivering therapy very specifically to active sites of disease.

Even coronary disease, in the absence of angioplasty, results in patches of denuded endothelium. (Davies et al., Br. Heart J., 60:459-64 (1988), which is incorporated herein by reference.) These patches offer attachment sites for RBCs carrying a therapeutic agent.

There is also a potential application in the treatment of solid tumors. It is well known that tumors are active sites of angiogenesis, small blood vessel formation. This process is necessary to tumor growth. It may be that nascent blood vessels in the tumor bed are open to blood flow before their endothelial lining is complete. Targeted RBCs may then attach to the walls of such vessels and deliver therapeutic agents which may then permeate surrounding tumor tissue through the immature vessel wall.

As noted above, a great advantage of the present technique is that high local concentrations of drugs can be achieved, without the toxicity that accompanies systemic use. It is envisioned that certain drugs could be developed specifically for use with the present vehicle. Such drugs could be too toxic for systemic use but very potent if delivered specifically to sites of disease activity.

The embodiments of the present invention recited herein are intended to be merely exemplary and those skilled in the art will be able to make numerous variations and modifications to it without departing from the spirit of the present invention. All such variations and modifications are intended to be within the scope of the present invention as defined by the claims appended hereto.

What is claimed is:

1. A method for impeding the deposition of platelets onto a recently exposed subendothelial surface in a patient, said method comprising the step of introducing into the bloodstream of the patient an effective amount of a multispecific antibody, said multispecific antibody comprising a first antigen binding site and a second antigen binding site, said first antigen binding site being directed against a surface marker of red blood cells, said second antigen binding site being directed against a subendothelial epitope.

2. The method as claimed in claim 1 wherein said subendothelial epitope is an epitope of a compound selected from the group consisting of collagen, elastin, laminin, and fibronectin.

3. The method as claimed in claim 1 wherein said surface marker of red blood cells is selected from the group consisting of the D antigen of the Rh blood group, glycophorin A, glycophorin B, and Band 3.

4. The method as claimed in claim 1 wherein said introducing step comprises injecting said effective amount of said multispecific antibody into the bloodstream of the patient.

5. The method as claimed in claim 1 wherein said injecting step is performed at about the time the subendothelial surface is exposed.

6. The method as claimed in claim 1 wherein said multispecific antibody is a bispecific antibody.

7. The method as claimed in claim 6 wherein said bispecific antibody is a bivalent bispecific antibody.

8. The method as claimed in claim 1 wherein said multispecific antibody is a mixture of two or more multi specific antibodies differing in at least one of their respective first antigen binding sites so as to bind to a variety of red blood cell surface markers and their respective second antigen binding sites so as to bind to a variety of subendothelial epitopes.

9. A method for binding red blood cells of a patient to an exposed subendothelial surface in said patient, said method comprising the step of introducing into the bloodstream of the patient an effective amount of a multispecific antibody, said multispecific antibody comprising a first antigen binding site and a second antigen binding site, said first antigen binding site being directed against a surface marker of red blood cells, said second antigen binding site being directed against a subendothelial epitope.

10. The method as claimed in claim 9 wherein said subendothelial epitope is an epitope of a compound selected from the group consisting of collagen, elastin, laminin, and fibronectin.

11. The method as claimed in claim 9 wherein said surface marker of red blood cells is selected from the group consisting of the D antigen of the Rh blood group, glycophorin A, glycophorin B, and Band 3.

12. The method as claimed in claim 9 wherein said introducing step comprises injecting said effective amount of said multispecific antibody into the bloodstream of the patient.

13. The method as claimed in claim 9 wherein the exposed endothelial surface is formed by an angioplasty and wherein said injecting step is performed at about the time of said angioplasty.

14. The method as claimed in claim 9 wherein said multispecific antibody is a bispecific antibody.

15. The method as claimed in claim 14 wherein said bispecific antibody is a bivalent bispecific antibody.

16. The method as claimed in claim 9 wherein said multispecific antibody is a mixture of two or more multispecific antibodies differing in at least one of their respective first antigen binding sites so as to bind to a variety of red blood cell surface markers and their respective second antigen binding sites so as to bind to a variety of subendothelial epitopes.

17. A method for targeted delivery of a therapeutic agent to an exposed subendothelial surface in a patient, said method comprising the step of introducing into the bloodstream of the patient a quantity of treated red blood cells, the treated red blood cells being adapted to bind to exposed subendothelial surfaces and having a therapeutic agent removably coupled thereto.

18. The method as claimed in claim 17 wherein the treated red blood cells comprise a red blood cell, a first multispecific antibody, a second multispecific antibody and a therapeutic agent, said first multispecific antibody having a first antigen binding site bound to a red blood cell surface marker and a second antigen binding site directed against a subendothelial epitope, said second multispecific antibody having a first antigen binding site bound to a red blood cell surface marker and a second antigen binding site removably bound to said therapeutic agent.

19. The method as claimed in claim 18 wherein said first antigen binding site of said first multispecific antibody and said first antigen binding site of said second multispecific antibody are bound to the same type of red blood cell surface marker.

20. The method as claimed in claim 18 wherein said first antigen binding site of said first multispecific antibody and said first antigen binding site of said second multispecific antibody are bound to different types of red blood cell surface markers.

21. The method as claimed in claim 17 wherein said subendothelial epitope is an epitope of a compound selected from the group consisting of collagen, elastin, laminin, and fibronectin.

22. The method as claimed in claim 17 wherein said red blood cell surface marker is selected from the group consisting of the D antigen of the Rh blood group, glycophorin A, glycophorin B and Band 3.

23. The method as claimed in claim 17 wherein said introducing step comprises injecting said effective quantity of treated red blood cells into the bloodstream of the patient.

24. The method as claimed in claim 17 wherein each of said first and second multispecific antibodies is a bispecific antibody.

25. The method as claimed in claim 24 wherein each of said first and second multispecific antibodies is a bivalent bispecific antibody.

26. The method as claimed in claim 17 wherein said therapeutic agent is selected from the group consisting of growth factors for promoting endothelialization, cytotoxic or cytostatic agents for inhibiting cell proliferation in the neointima, and immunosuppressive agents.

27. A method for targeted delivery of a therapeutic agent to an exposed subendothelial surface in a patient, said method comprising the steps of:
(a) obtaining a sample of red blood cells from the patient;
(b) adding to the sample of red blood cells a first multispecific antibody, a second multispecific antibody and a therapeutic agent, said first multispecific antibody having a first antigen binding site directed against a red blood cell surface marker and a second antigen binding site directed against a subendothelial epitope, said second multispecific antibody having a first antigen binding site directed against a red blood cell surface marker and a second antigen binding site directed against said therapeutic agent; and
(c) introducing the product of step (b) into the bloodstream of the patient.

28. The method as claimed in claim 27 wherein said first multispecific antibody, said second multispecific antibody and said therapeutic agent are added to the sample sequentially.

29. The method as claimed in claim 27 wherein said first multispecilic antibody, said second multispecific antibody and said therapeutic agent are added to the sample simultaneously.

30. The method as claimed in claim 27 wherein said therapeutic agent is selected from the group consisting of growth factors for promoting endothelialization, cytotoxic or cytostatic agents for inhibiting cell proliferation in the neointima, and immunosuppressive agents.

31. A multispecific antibody, said multispecific antibody comprising a first antigen binding site and a second antigen binding site, said first antigen binding site being directed against a red blood cell surface marker, said second antigen binding site being directed against a subendothelial epitope.

32. A multispecific antibody, said multispecific antibody comprising a first antigen binding site and a second antigen binding site, said first antigen binding site being directed against a red blood cell surface marker, said second antigen binding site being directed against a therapeutic agent for treating an exposed subendothelial surface.

33. The combination of a red blood cell, a first multispecific antibody, a second multispecific antibody and a therapeutic agent, said first multispecific antibody comprising a first antigen binding site directed against a red blood cell surface marker and a second antigen binding site directed against a subendothelial epitope, said second multispecific antibody comprising a first antigen binding site directed against a red blood cell surface marker and a second antigen binding site directed against said therapeutic agent.

34. The combination of claim 33 wherein said first antigen binding site of said first multispecific antibody and said first antigen binding site of said second multispecific antibody are directed to the same type of red blood cell surface marker.

35. The combination of claim 33 wherein said first antigen binding site of said first multispecific antibody and said first antigen binding site of said second multispecific antibody are directed to different types of red blood cell surface markers.

36. The combination of claim 33 wherein said therapeutic agent is selected from the group consisting of growth factors for promoting endothelialization, cytotoxic or cytostatic agents for inhibiting cell proliferation in the neointima, and immunosuppressive agents.
